SEQUENCE LISTING (2 sequences)

SEQ ID NO: 1 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEALLSKAVEIEEQTKRLLRGMELIVSQVHPETKENEIYPVWSGLPSLQMADEESRLSAYYNLLHCLRRDSHKIDNYLKLLKCRIIHNNNC

SEQ ID NO: 2 - protein, 199 aa, Artificial Sequence
  Homo sapiens Gly129 repalced with Arg
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEALLSKAVEIEEQTKRLLRRMELIVSQVHPETKENEIYPVWSGLPSLQMADEESRLSAYYNLLHCLRRDSHKIDNYLKLLKCRIIHNNNC

(19) United States
(12) Patent Application Publication
(10) Pub. No.: US 2016/0354444 A1
(43) Pub. Date: Dec. 8, 2016

(54) COMPOSITIONS AND METHODS FOR THE TREATMENT OF SEXUAL DYSFUNCTION

(71) Applicant: Vijaykumar Rajasekhar, Apple Valley, CA (US)

(72) Inventor: Vijaykumar Rajasekhar, Apple Valley, CA (US)

(21) Appl. No.: 15/173,398

(22) Filed: Jun. 3, 2016

Related U.S. Application Data
(60) Provisional application No. 62/170,989, filed on Jun. 4, 2015.

(51) Int. Cl. A61K 38/22; A61K 9/14; A61K 47/48; A61K 9/00

(52) U.S. Cl. CPC A61K 38/2257; A61K 9/0014; A61K 9/0073; A61K 9/14; A61K 47/48023

(57) ABSTRACT

Described herein are pharmaceutical compositions for the treatment of sexual dysfunction in an individual. In some embodiments, the pharmaceutical composition includes a prolactin variant having a glycine residue at position 129 substituted with an amino acid other than glycine. In one embodiment, the amino acid is arginine. In one embodiment, the prolactin variant further comprises an N-terminal deletion. In one embodiment, the prolactin variant is conjugated to a H(OCH₂CH₂)ₙOH molecule (e.g., n equals any number from 1 to 6). In some embodiments, the pharmaceutical composition is delivered to the individual by a microneedle patch or an inhalable formulation.

### CROSS-REFERENCE TO RELATED APPLICATIONS

This patent application claims priority to U.S. provisional patent application Ser. No. 62/170,989, filed on Jun. 4, 2015, which is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### FIELD OF THE INVENTION

The present invention relates generally to the field of sexual dysfunction and treatment, and more particularly to compositions and methods that are useful in reducing the actions of prolactin to treat sexual dysfunction, including but not limited to reducing the length of sexual refractory period in humans. The compositions and methods described here may also be useful for treating other sexual dysfunctions including but not limited to: decreased orgasm; anorgasmia; decreased libido; erectile dysfunction; premature ejaculation; and drowsiness, sleepiness, or fatigue associated with orgasm or sexual activity.

### BACKGROUND

A major complaint of aging is the increasing length of sexual refractory period in men. The sexual refractory period is the recovery phase after orgasm during which it is physiologically impossible for a man to have additional orgasms or additional erections.

In contrast to women (who can achieve multiple sequential orgasms throughout life), men typically have a sexual refractory period (i.e., the time in between erectile events) that lengthens with age. By the time men hit the fourth and fifth decade of life, many have recognized that their erectile function has changed; the ability to maintain an erection during sex has diminished, and the refractory period begins to increase.

In young adult years, the average male refractory period ranges from fifteen to twenty minutes. In some elderly populations, the male refractory period increases to hours or even days. In some elderly men, the refractory period may extend up to twenty to forty hours.

Erection is initiated by some form of sexual stimulus, which causes the release of nitric oxide (NO) from cavernosa nerves in the penis. NO diffuses into smooth muscle cells of both the corpus cavernosa and the penile arterial system, where it activates the enzyme guanylyl cyclase (GC), which synthesizes cyclic guanosine monophosphate (cGMP), an intracelluar second-messenger molecule. Activation of cGMP causes (1) relaxation of the smooth muscle cells within the media of the penile arterial vessels (which leads to arterial vasodilatation and an increase in blood flow to the penis), and (2) relaxation of the smooth muscle cells within the cavernosa bodies, which opens the cavernosa sinusoids to provide a place to store the increase in penile blood flow.

As the body ages, the tissues that contain smooth muscle (e.g., bladder, peripheral vascular system, penis) display a progressive loss of the parenchymal smooth muscle cells, which are replaced by collagen fibers. The loss of smooth muscle cells in the penis means that the remaining smooth muscle mass may not function as well as it did previously and, from a physiologic point of view, may not be able to relax and store blood as well as it did with its full complement of smooth muscle cells. A man needs to lose approximately 15% of corporeal smooth muscle mass before leakage of blood occurs from the penis. Because this programmed cell loss (i.e., apoptosis) with aging is thought to be a genetically determined event, the recognition of an increase in the refractory period or the inability to maintain an erection will occur at a different age for each individual. This explains why the majority of men, regardless of age, have evidence of venous leakage or cavernosa venoocclusive dysfunction (CVOD) when evaluated as a result of complaints of erectile dysfunction (ED).

The inability to attain and maintain an erection (i.e., erectile dysfunction) is the result of a dynamic imbalance between inflow and outflow of blood within the cavernosa bodies. As there is increased loss of function in corporeal smooth muscle cells, CVOD increases leading to an increase of refractory period due to decreased ability to relax the smooth muscles needed to open the cavernosa sinusoids to store blood and thus a longer period of time is needed to fill the sinusoids to create subsequent erections.

As refractory period worsens, a patient eventually suffers from ED when (1) the venous outflow, which is dependent on the amount of corporeal smooth muscle apoptosis, reaches the critical stage in which the arterial inflow is insufficient to compensate for the progressive increase in venous leakage; or (2) the inflow becomes so restricted that it is incapable of overcoming the amount of venous leakage that is already present.

Phosphodiesterase type 5 (PDE5) inhibitors treat ED by blocking the degradative action of cGMP-specific PDE5 on cGMP in the smooth muscle cells lining the blood vessels supplying the corpus cavernosum of the penis. PDE5 inhibitors inhibit this activity permitting cGMP to remain active as a second messenger, leading to the physiological outcome (e.g., erection). Examples of PDE5 inhibitors include: sildenafil (i.e., Viagra), tadalafil (i.e., Cialis), and vardenafil (i.e., Levitra). PDE5 inhibitors have successfully been used to treat ED, defined as the inability to initiate a primary erection. Despite the success of PDE5 inhibitors to help patients achieve a primary erection, these inhibitors do not sufficiently reduce a man's refractory period after an initial orgasm or ejaculation. Thus, even with PDE5 inhibitors, it is difficult for a man to have continued erections or continued orgasms.

Due to the male refractory period and inability of men to maintain intercourse after male ejaculation and orgasm, women tend to experience orgasm with less frequency during sexual intercourse. Described alternatively, men tend to reach orgasm before women, and due to the male refractory period, sexual intercourse may be terminated before a woman attains one or more desired orgasms. Thus, the male refractory period plays a significant role in sexual health, intimacy, and relationships.

One in five men suffer from premature ejaculation (PE), regardless of age. Due to the male refractory period, PE further reduces the ability of both partners to achieve orgasm. Once a man with PE has orgasm, intercourse is terminated prematurely and cannot resume immediately due to the male refractory period.

Male refractory periods, which (1) lengthen with age and (2) impact continued sexual activity in premature ejaculation, represent major unmet medical needs, with a potential market expected to exceed $4 billion annually.

The global ED market in 2012 was $4.3 billion. This market does not include men who are contraindicated for taking PDE5 inhibitors because they are taking nitrates. The number of men who could benefit from a solution that reduces male refractory period will likely exceed the number who could benefit from PDE5 inhibitors for ED, because physiologically, refractory periods begin to lengthen in men before clinical symptoms of ED even present. Said alternatively, out of the large pool of men with lengthened refractory period, only a portion may develop ED. While many men in this population suffer from ED, many more are impacted by lengthened refractory period and an inability to have sequential orgasms.

Thus, there is a need for new and useful compositions and methods for the treatment of sexual dysfunction. This invention provides such new and useful compositions and methods.

### SUMMARY

The present disclosure is directed to compositions and methods for treating sexual dysfunction. One aspect of the present disclosure is directed to a pharmaceutical composition for the treatment of sexual dysfunction in an individual. In some embodiments, a pharmaceutical composition for the treatment of sexual dysfunction in an individual includes a prolactin variant having a glycine residue at position 129 substituted with an amino acid other than glycine. In some embodiments, the amino acid is arginine. In some embodiments, the prolactin variant further comprises an N-terminal deletion. In some embodiments, the prolactin variant is conjugated to a H(OCH₂CH₂)ₙOH molecule, where n equals any number from one to six.

In some embodiments, the pharmaceutical composition is delivered to the 120 individual by a microneedle patch.

In some embodiments, administration of the prolactin variant effectively reduces an effect of 5 to 15 ng/mL of prolactin in the plasma of the individual.

In some embodiments, the pharmaceutical composition is an inhalable composition. In some embodiments, the inhalable composition has a concentration between 1,400 to 21,500 ng/mL of the prolactin variant. In some embodiments, the inhalable composition is a powder composition. In some embodiments, the pharmaceutical composition further includes one or more of lactose and sodium phosphate as a pharmaceutically acceptable excipient.

In some embodiments, the sexual dysfunction comprises one or more of the following: erectile dysfunction, prolonged sexual refractory period, premature ejaculation, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, and drowsiness with orgasm or sexual activity.

One aspect of the present disclosure is directed to a pharmaceutical composition for the treatment of sexual dysfunction in an individual. In some embodiments, a pharmaceutical composition for the treatment of sexual dysfunction in an individual includes a prolactin suppressor. In some embodiments, the prolactin suppressor is a prolactin variant in which a glycine residue at position 129 is substituted with arginine. In some embodiments, the prolactin suppressor is conjugated to a H(OCH₂CH₂)ₙOH molecule, where n equals any number from 1 to 6. In some embodiments, the pharmaceutical composition further comprises one or more of zinc or a PDE5 inhibitor.

In some embodiments, the sexual dysfunction comprises one or more of the following: erectile dysfunction, prolonged sexual refractory period, premature ejaculation, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, and drowsiness with orgasm or sexual activity.

In some embodiments, the prolactin suppressor comprises one or more of the following: prolactin variants, prolactin receptor antagonists, truncated prolactin receptors, prolactin receptor inhibitors, serum prolactin binding proteins, soluble isoforms of the prolactin receptor, zinc, dopamine agonists, prolactin antibodies, prolactin receptor antibodies, growth hormone, growth hormone variants, truncated growth hormone receptors, placental lactogen, placental lactogen variants, truncated lactogen receptors, choriomammotropin, proliferin, somatolactin, estrogen, estrogen variants, progesterone, and progesterone variants.

In some embodiments, the prolactin suppressor directly or indirectly binds a prolactin receptor.

In some embodiments, the prolactin suppressor is a soluble form of a prolactin receptor isoform that results from alternative splicing of a prolactin receptor primary mRNA transcript from exon 7 to exon 11.

In some embodiments, the prolactin suppressor comprises a mutation in one of a prolactin receptor activation domain and a prolactin receptor heterodimerization domain

One aspect of the disclosure is directed to a method for treating sexual dysfunction. In some embodiments, a method for treating sexual dysfunction in a patient includes administering to the patient an effective amount of one or more prolactin (PRL) suppressors. In some embodiments, sexual dysfunction includes one or more of the following: erectile dysfunction, prolonged sexual refractory period, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, and drowsiness with orgasm or sexual activity. In some embodiments, PRL suppressors include one or more of the following: PRL variants, prolactin receptor (PRLR) antagonists, truncated PRLRs, PRLR inhibitors, serum PRL binding proteins, soluble isoforms of the PRLR, zinc, dopamine agonists, PRL antibodies, PRLR antibodies, growth hormone, growth hormone variants, truncated growth hormone receptors, placental lactogen, placental lactogen variants, truncated lactogen receptors, choriomammotropin, proliferin, somatolactin, other proteins or variants of the somatotropin family, estrogen, estrogen variants, progesterone, and progesterone variants.

One aspect of the disclosure is directed to a method of reducing sexual refractory period. In some embodiments, a method for reducing sexual refractory period in a patient includes administering to the patient an effective amount of one or more PRL suppressors. In some embodiments, the PRL suppressor includes a PRL variant. In some embodiments, the PRL suppressor includes a PRLR antagonist. In some embodiments, the PRL suppressor includes a PRLR inhibitor. In some embodiments, the PRL suppressor includes a serum PRL binding protein. In some embodiments, the PRL suppressor includes a soluble isoform of the PRLR. In some embodiments, the PRL suppressor includes a serum PRL binding protein or soluble isoform of the PRLR that preferentially binds PRL over growth hormone. In some embodiments, the PRL suppressor includes zinc. In some embodiments, the PRL suppressor includes a dopamine agonist. In some embodiments, the PRL suppressor includes a PRL antibody or PRLR antibody. In some embodiments, the PRL suppressor includes one or more steroids, such as estrogen, progesterone, or variants of progesterone or estrogen. In some embodiments, the PRL suppressor includes growth hormone, variants of growth hormone, and/or truncated growth hormone receptors. In some embodiments, the PRL suppressor includes lactogen, lactogen variants, and other members and variants of the somatotropin family.

One aspect of the disclosure is directed to a method for increasing the frequency and/or duration of orgasm. In some embodiments, a method for increasing the frequency and/or duration of orgasm in a patient includes administering to the patient an effective amount of one or more PRL suppressors. In some embodiments, the PRL suppressor includes a PRL variant. In some embodiments, PRL suppressor includes a PRLR antagonist. In some embodiments, the PRL suppressor includes a PRLR inhibitor. In some embodiments, the PRL suppressor includes a serum PRL binding protein. In some embodiments, the PRL suppressor includes a soluble isoform of the PRLR. In some embodiments, the PRL suppressor includes a serum PRL binding protein or soluble isoform of the PRLR that preferentially bind PRL over growth hormone. In some embodiments, the PRL suppressor includes zinc. In some embodiments, the PRL suppressor includes a dopamine agonist. In some embodiments, the PRL suppressor includes a PRL antibody or PRLR antibody. In some embodiments, the PRL suppressor includes one or more steroids, such as estrogen, progesterone, or variants of progesterone or estrogen. In some embodiments, the PRL suppressor includes growth hormone or variants of growth hormone, and/or truncated growth hormone receptors. In some embodiments, the PRL suppressor includes lactogen, lactogen variants, and other members and variants of the somatotropin family. I

In some embodiments, the PRL suppressor is a PRL variant in which a glycine residue at position 129 is substituted with another amino acid. In some embodiments, the amino acid is selected from the group consisting of: valine, leucine, isoleucine, serine, threonine, proline, tyrosine, cysteine, methionine, arginine, histidine, tryptophan, phenylalanine, lysine, asparagine, glutamine, aspartic acid and glutamic acid. In some embodiments, the amino acid is arginine. In some embodiments, the PRL suppressor is G129R hPRL.

In some embodiments, the PRL suppressor includes a deletion of at least the first 9 N-terminal residues. In some embodiments, the PRL suppressor includes a deletion of exactly the first 9 N-terminal residues or a deletion of more than the first 9 N-terminal residues. In some embodiments, the PRL variant is Δ1-9-G129R-hPRL. In some embodiments, the PRL variant is Δ1-14-G129R-hPRL.

In some embodiments, the PRL suppressor is a molecule that directly binds PRL. In some embodiments, the molecule is a soluble PRLR isoform that results from alternative splicing of the PRLR primary mRNA transcript from exon 7 to exon 1. In some embodiments, the PRL suppressor is a molecule that preferentially binds PRL over growth hormone. In some embodiments, the PRL suppressor is a PRL binding protein. In some embodiments, the PRL suppressor is monoclonal antibody LFA102.

One aspect of the disclosure is directed to a method for treating sexual dysfunction. In some embodiments, a method for treating sexual dysfunction in a patient includes administering to the patient an effective amount of one or more PRL suppressors alone or in combination with one or more PDE5 inhibitors. In some embodiments, the sexual dysfunction includes one or more of the following: erectile dysfunction, prolonged sexual refractory period, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, and drowsiness with orgasm or sexual activity. In some embodiments, PRL suppressor includes one or more of the following: PRL variants, PRLR antagonists, truncated PRLRs, PRLR inhibitors, serum PRL binding proteins, soluble isoforms of the PRLR, zinc, dopamine agonists, anti-PRL antibodies, anti-PRLR antibodies, growth hormone, growth hormone variants, truncated growth hormone receptors, placental lactogen, placental lactogen variants, truncated lactogen receptors, choriomammotropin, proliferin, somatolactin, other proteins or variants of the somatotropin family, estrogen, estrogen variants, progesterone, and progesterone variants. In some embodiments, the PDE5 inhibitors include Acetildenafil, Aildenafil, Avanafil, Benzamidenafil, Icariin, Lodenafil, Mirodenafil, Nitrosoprodenafil, Sildenafil, Sulfoaildenafil, Tadalafil, Udenafil, Vardenafil, or Zaprinast.

In some embodiments, the sexual dysfunction is a side effect from a medication. In some embodiments, the medication is an anti-depressant medication. In some embodiments, the anti-depressant medication is an SSRI (Selective Serotonin Reuptake Inhibitor).

In some embodiments, one or more PRL suppressors are administered simultaneously or sequentially with one or more PDE5 inhibitors. In some embodiments, the PRL suppressor is administered orally, sublingually, buccally, subcutaneously, rectally, transdermally, or inhaled via pulmonary delivery. In some embodiments, the PDE5 inhibitor is administered orally, sublingually, buccally, subcutaneously, rectally, transdermally, or inhaled via pulmonary delivery. In some embodiments, one or more PRL suppressors are administered by pulmonary delivery simultaneously or sequentially with one or more PDE5 inhibitors. In some embodiments, one or more PDE5 inhibitors are delivered by pulmonary delivery. In some embodiments, one or more PDE5 inhibitors are delivered by a non-pulmonary route, such as orally, sublingually, buccally, subcutaneously, rectally, transdermal routes, or other non-pulmonary routes. In some embodiments, pulmonary delivery to a patient is by a pulmonary device inserted onto, into, or around the month or nose. In some embodiments, zinc is delivered with one or more PRL suppressors that are administered by pulmonary delivery simultaneously or sequentially with one or more PDE5 inhibitors.

In some embodiments, a pharmaceutical composition includes particles that are delivered from said pulmonary device. In some embodiments, the device is selected from the group consisting of: a nebulizer, a metered-dose inhaler, and a dry powder inhaler. In some embodiments, the device is a dry powder inhaler. In some embodiments, the particle further includes a buffer selected from the group consisting of: sodium phosphate, TRIS, maleate, and glycine. In some embodiments, the buffer is sodium phosphate. In some embodiments, the particle comprises about 93.5% PRL suppressor and about 6.5% sodium phosphate. In some embodiments, PRL suppressor is pegylated. In some embodiments, the PRL suppressor is a PRLR antagonist, PRL variant, or PRLR inhibitor. In some embodiments, the PRL suppressor is a PRL variant in which a glycine residue at position 129 is substituted with another amino acid or there is a deletion of at least the first 9 N-terminal residues. In some embodiments, the PRL variant includes G129R-hPRL. In some embodiments, PRL variant includes pegylated G129R-hPRL. In some embodiments, the PRL suppressor is a PRL variant in which a glycine residue at position 129 is substituted with another amino acid and there is a deletion of at least the first 9 N-terminal residues. In some embodiments, the PRL variant includes Δ1-9-G129R-hPRL. In some embodiments, the PRL variant includes pegylated Δ1-9-G129R-hPRL. In some embodiments, the PRL suppressor is a serum PRL binding protein or a soluble isoform of the PRLR. In some embodiments, the PRL suppressor preferentially binds PRL over growth hormone.

In some embodiments, the PRL suppressor is a soluble form PRLR isoform that results from alternative splicing of the PRLR primary mRNA transcript from exon 7 to exon 11.

In some embodiments, the PRL suppressor is a PRL antibody or PRLR antibody. In some embodiments, the PRL suppressor is monoclonal antibody LFA102.

One aspect of the disclosure is directed to a pharmaceutical composition. In some embodiments, a pharmaceutical composition in a unit dosage form comprising a dry powder suitable for pulmonary administration by a patient to the deep lung includes a PRL suppressor with or without pegylation and a buffer. In some embodiments, the PRL suppressor is a PRL variant in which a glycine residue at position 129 is substituted with another amino acid or there is a deletion of at least the first 9 N-terminal residues. In some embodiments, the PRL variant includes G129R-hPRL. In some embodiments, the PRL variant includes pegylated G129R-hPRL. In some embodiments, the PRL suppressor is a PRL variant in which a glycine residue at position 129 is substituted with another amino acid and there is a deletion of at least the first 9 N-terminal residues. In some embodiments, the PRL variant includes Δ1-9-G129R-hPRL. In some embodiments, the PRL variant includes pegylated Δ1-9-G129R-hPRL. In some embodiments, the PRL suppressor is a serum PRL binding protein or a soluble isoform of the PRLR. In some embodiments, the PRL suppressor preferentially binds PRL over growth hormone. In some embodiments, the PRL suppressor is a soluble form PRLR isoform that results from alternative splicing of the PRLR primary mRNA transcript from exon 7 to exon 11. In some embodiments, the PRL suppressor is a PRL antibody or PRLR antibody. In some embodiments, the PRL suppressor is monoclonal antibody LFA102.

In some embodiments, the buffer is selected from the group consisting of sodium phosphate, TRIS, maleate, and glycine. In some embodiments, the unit dosage form is a capsule. In one embodiment, the capsule includes a unit dosage of 3.0 mg of the pharmaceutical composition. In one embodiment, the capsule includes a unit dosage of 4.8 mg of the pharmaceutical composition. In one embodiment, the capsule includes a unit dosage of 6.0 mg of the pharmaceutical composition. In one embodiment, the capsule includes a unit dosage of 9.0 mg of the pharmaceutical composition. In one embodiment, the capsule includes a unit dosage of 12.0 mg of the pharmaceutical composition. In one embodiment, the capsule includes a unit dosage of 15.1 mg of the pharmaceutical composition. In one embodiment, the capsule contains a unit dosage of 21.1 mg of the pharmaceutical composition.

One aspect of the disclosure is directed to a method of treating sexual dysfunction. In some embodiments, a method of treating sexual dysfunction includes administering to the patient an effective amount of one or more pegylated PRL suppressors. In some embodiments, the sexual dysfunction includes one or more of the following: erectile dysfunction, prolonged sexual refractory period, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, and drowsiness with orgasm or sexual activity. In some embodiments, the PRL suppressor includes one or more of the following: PRL variants, PRLR antagonists, truncated PRLRs, PRLR inhibitors, serum PRL binding proteins, soluble isoforms of the PRLR, zinc, dopamine agonists, anti-PRL antibodies, anti-PRLR antibodies, growth hormone, growth hormone variants, truncated growth hormone receptors, placental lactogen, placental lactogen variants, truncated lactogen receptors, choriomammotropin, proliferin, somatolactin, proteins or variants of the somatotropin family, estrogen, estrogen variants, progesterone, and progesterone variants.

One aspect of the disclosure is directed to a method of producing a pegylated PRL suppressor. In some embodiments, a method of producing a pegylated PRL suppressor to treat sexual dysfunction includes pegylating a PRL suppressor; applying the pegylated PRL suppressor to a cation exchange chromatography column; and eluting the pegylated PRL suppressor. In some embodiments, the sexual dysfunction includes one or more of the following: erectile dysfunction, prolonged sexual refractory period, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, and drowsiness with orgasm or sexual activity. In some embodiments, the PRL suppressor includes one or more of the following: PRL variants, PRLR antagonists, truncated PRLRs, PRLR inhibitors, serum PRL binding proteins, soluble isoforms of the PRLR, zinc, dopamine agonists, anti-PRL antibodies, anti-PRLR antibodies, growth hormone, growth hormone variants, truncated growth hormone receptors, placental lactogen, placental lactogen variants, truncated lactogen receptors, choriomammotropin, proliferin, somatolactin, other proteins or variants of the somatotropin family, as well as estrogen, estrogen variants, progesterone, and progesterone variants.

In some embodiments, the PRL suppressor is a PRL variant. In some embodiments, the PRL variant is PRL with a glycine residue at position 129 substituted with another amino acid or a deletion of at least the first 9 N-terminal residues. In some embodiments, the PRL variant includes G129R-hPRL.

In some embodiments, the PRL variant is PRL with a glycine residue at position 129 is substituted with another amino acid and there is a deletion of at least the first 9 N-terminal residues. In some embodiments, the PRL variant includes Δ1-9-G129R-hPRL.

In some embodiments, the PRL variant is conjugated to one or more chemical groups that increase the actual molecular weight of the PRL variant to between about 22 and about 200 kilodaltons. In some embodiments, the chemical group is polyethylene glycol. In some embodiments, the PRL variant is conjugated to between about one to about six molecules of polyethylene glycol. In some embodiments, the PRL suppressor is a serum PRL binding protein or a soluble isoform of the PRLR. In some embodiments, the PRL suppressor preferentially binds PRL over growth hormone. In some embodiments, the PRL suppressor is a soluble form PRLR isoform that results from alternative splicing of the PRLR primary mRNA transcript from exon 7 to exon 11. In some embodiments, the PRL suppressor is a PRL antibody or PRLR antibody. In some embodiments, the PRL suppressor is monoclonal antibody LFA102.

One aspect of the disclosure is directed to an inhalable powder formulation. In some embodiments, an inhalable powder formulation includes a PRL suppressor as an active ingredient for preventing or treating a sexual dysfunction condition. In some embodiments, the sexual dysfunction condition selected from the group consisting of one or more of the following: erectile dysfunction, prolonged sexual refractory period, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, and drowsiness with orgasm or sexual activity

In some embodiments, the inhalable powder formulation further includes a pharmaceutically acceptable excipient. In some embodiments, the pharmaceutically acceptable excipient is selected from the group consisting of: monosaccharides such as glucose and arabinose; disaccharides such as lactose, saccharose and maltose; polysaccharides such as sorbitol, mannitol and xylitol; salts such as sodium chloride, calcium carbonate, or sodium phosphate; sodium bicarbonate; amino acids; peptides; polymers; lipids; and a mixture thereof. In some embodiments, the inhalable powder formulation includes a PRL suppressor (such as a PRL variant such as G129R-hPRL or Δ1-9-G129R-hPRL), lactose, and dipalmitoyl phosphatidylcholine (DPPC). In some embodiments, the inhalable powder formulation includes a PRL suppressor (such as a PRL variant such as G129R-hPRL or Δ1-9-G129R-hPRL), lactose, and dipalmitoyl phosphatidylcholine (DPPC) in the ratio of 1:0.5 to 1.5:1.5 by weight.

One aspect of the disclosure is directed to a method of preventing or treating sexual dysfunction. In some embodiments, a method for preventing or treating sexual dysfunction in a subject includes administering a therapeutically effective amount of an inhalable powder formulation including a PRL suppressor (such as a PRL variant such as G129R-hPRL or Δ1-9-G129R-hPRL) to the subject in need thereof.

One aspect of the disclosure is directed to an inhalable powder formulation for preventing or treating a sexual dysfunction condition. In some embodiments, an inhalable powder formulation for preventing or treating a sexual dysfunction condition includes a PRL suppressor (such as a PRL variant such as G129R-hPRL or Δ1-9-G129R-hPRL) as an active ingredient. In some embodiments, the inhalable powder formulation includes a PRL suppressor (such as a PRL variant such as G129R-hPRL or Δ1-9-G129R-hPRL), sodium phosphate, and dipalmitoyl phosphatidylcholine (DPPC).

### BRIEF DESCRIPTION OF THE DRAWINGS

FIG. 1A illustrates the amino acid sequence of Human prolactin.

FIG. 1B illustrates the amino acid sequence of Human prolactin variant (G129R hPRL) by substituting Glycine with Arginine at position 129.

FIG. 2A illustrates a schematic representation of the cloning and construction of the expression plasmid pUCIG-MT-hPRL-cDNA.

FIG. 2B illustrates the plasmid map and general strategy of PCR-directed mutagenesis.

### DETAILED DESCRIPTION

The following description of the preferred embodiments of the invention is not intended to limit the invention to these preferred embodiments, but rather to enable any person skilled in the art to make and use this invention. Disclosed herein are compositions and methods for the treatment of sexual dysfunction.

Sexual dysfunction includes, but is not limited to: prolonged sexual refractory period; premature ejaculation; erectile dysfunction; anorgasmia; decreased frequency or duration of orgasms; decreased sexual desire and libido; hypoactive sexual desire disorder; reduced penile erections; delayed ejaculation; drowsiness, fatigue, or sleepiness associated with orgasm and sexual activity; and other conditions known generally as sexual dysfunction.

As described elsewhere herein, sexual dysfunction may be treated by inhibiting the action of prolactin (PRL). PRL is an anterior pituitary hormone involved in a wide spectrum of biological activities, among which are those related to lactation and reproduction. PRL is released at orgasm in both women and men.

Orgasm (i.e., sexual climax) is the sudden discharge of accumulated sexual excitement during the sexual response cycle, resulting in rhythmic muscular contractions in the pelvic region characterized by sexual pleasure. Experienced by males and females, orgasms are controlled by the involuntary or autonomic nervous system. They are often associated with other involuntary actions, including muscle spasms in multiple areas of the body, and a general euphoric sensation. In males, orgasm is often accompanied with ejaculation. The period after orgasm is often a relaxing experience attributed to the release of the hormones oxytocin and PRL, as well as endorphins.

Plasma PRL concentrations are substantially increased for over one hour following orgasm in both men and women, but PRL concentrations are unchanged following sexual arousal without orgasm (Kruger et al. “Orgasm-induced prolactin secretion: feedback control of sexual drive?” Neuroscience and Biobehavioral Reviews 26 (2002): 31-44.). Further, it was found that exogenously administered PRL reliably suppresses erection in dogs (Aoki et al. “Suppression by Prolactin of the Electrically Induced Erectile Response through its Direct Effect on the Corpus Cavernous Penis in the Dog.” The Journal of Urology 154 (1996): 595-600.). Additionally, patients with prolactinoma (i.e., pituitary gland tumor with high levels of PRL secretion) are known to have well-documented sexual side effects (e.g., low libido, sexual dysfunction). Current drugs to treat prolactinomas (pituitary adenomas) are dopamine agonists, which are a type of PRL suppressor, as described elsewhere herein. dopamine and dopamine agonists inhibit central PRL secretion. Dopamine agonist drugs are 80-90% effective in pharmacologically treating prolactinoma. While cabergoline and other dopamine agonists are good candidates for managing prolactinomas, they are not good candidates for the treatment of prolonged sexual refractory period or other sexual dysfunctions because these drugs have an unfavorable side effect profile and take extended time to titrate to therapeutic levels.

Described herein are methods and compositions for reducing the effect of PRL in a patient's body in order to treat sexual dysfunctions, such as prolonged refractory period, or premature ejaculation. The effect of PRL may be reduced by one or more PRL suppressors.

As described herein, “prolactin” refers to the protein prolactin of any mammal, species, or organism. In one embodiment, “prolactin” refers to the protein prolactin derived from a mouse, human, or monkey.

PRL “suppressors” are defined as any elements, molecules, reagents, compounds, biologics, injectables, inhalants, therapeutics, and/or drugs, that are either natural or synthetic, that have the ability to directly or indirectly modulate the action of PRL in the mammalian body. For example, PRL suppressors may decrease the production or secretion of PRL centrally or peripherally, may interfere with PRL in the serum (e.g., via mechanisms such as PRL binding proteins (PRL-BP)), may interfere with PRL binding at the prolactin receptor (PRLR), or may directly bind PRLR and act as an agonist or antagonist. Such PRL suppressors function, for example, by binding PRL in the serum in order to reduce PRL's actions, by binding PRL in the serum to reduce the ability of PRL to bind to PRLRs, by competitive inhibition where PRL suppressors bind PRLR so that fewer PRL molecules can bind PRLR, by directly binding PRLR to act as antagonists that prevent the downstream actions of the PRLR, by reducing the number of PRLR, by directly or indirectly antagonizing the action of PRLR, by antagonizing the action of PRL at the PRLR, and/or by reducing the feedback of PRL from peripheral tissues back to the central nervous system.

PRL suppressors as described elsewhere herein or in the art, may be used to inhibit the effects of PRL, and in particular, may be used to treat sexual dysfunction, including prolonged refractory period, or refractory period with premature ejaculation.

As described herein, “prolactin suppressor” refers to any protein, DNA, RNA, drug, pharmaceutical, or any other biologic or chemical that inhibits the action of naturally occurring prolactin. In some embodiments, the prolactin suppressor is derived from any mammal, species, or organism. In one embodiment, the prolactin suppressor is derived from a mouse, human, or monkey.

PRL suppressors may include but are not limited to: PRLR antagonists, PRLR inhibitors, PRL variants, truncated PRLR (e.g., competes with endogenous PRLR for PRL binding), serum PRL binding proteins, soluble isoforms of the PRLR, dopamine agonists, antibodies specific for PRL, antibodies specific for PRLR, growth hormone (GH), GH variants, placental lactogen (PL), PL variants, other members of the somatotropin family, zinc, Estrogen, Estrogen variants, Progesterone, and Progesterone variants, as well as a number of other compounds that are either currently available, in development, or may be developed in the future that can manipulate the amount or action of PRL in the mammalian body. In some embodiments, molecules that are PRL variants antagonize the action of PRLR or antagonize the action of PRL at the PRLR and are therefore considered PRLR antagonists or PRLR inhibitors. In some embodiments, molecules or compounds that are not variants of PRL can also act as PRLR antagonists or PRLR inhibitors by either directly or indirectly antagonizing the action of PRLR. For example, GH variants can also act as PRLR antagonists.

The ability of such a PRL antagonist to antagonize the action of PRL at its receptor is defined as the ability of the molecule or compound to inhibit an effect mediated, under normal conditions, by PRL. For example, where PRL has an effect on penile erectile tissue to increase the sexual refractory period in a man, a PRL antagonist inhibits the effect on erectile tissue. In some embodiments, a PRL variant, which acts as an antagonist at the PRLR, is used for the treatment of sexual dysfunction. The term PRL variant refers to a form of PRL that has been structurally altered relative to its native form, including where the amino acid sequence of the native form has been altered by the insertion, deletion, and/or substitution of amino acids. In one embodiment, a PRL variant is a mutated form of human PRL (hPRL) in which the glycine amino acid at position 129 (G129) is substituted with another amino acid. For example, G129 of PRL may be substituted with arginine (G129R). In one embodiment, a PRL variant is Δ1-9-G129R-hPRL, wherein G129 is substituted for arginine and there is a deletion of the 9 N-terminal residues. In one embodiment, the PRL variant is Δ1-14-G129R-hPRL, wherein G129 is substituted for arginine and there is a deletion of the 14 N-terminal residues.

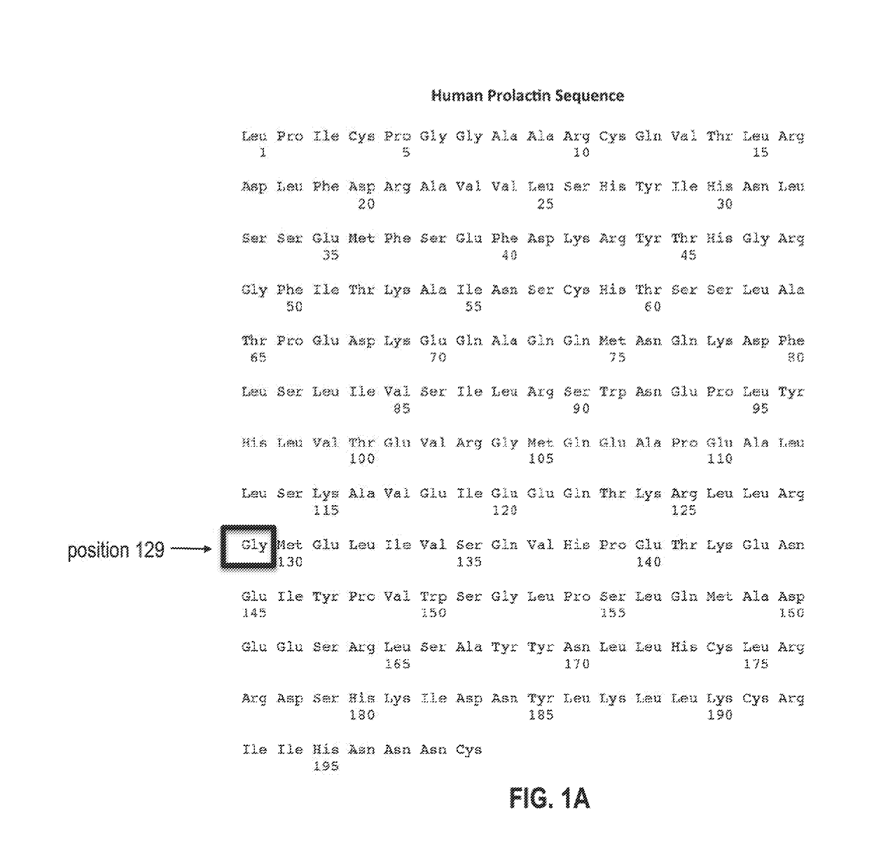

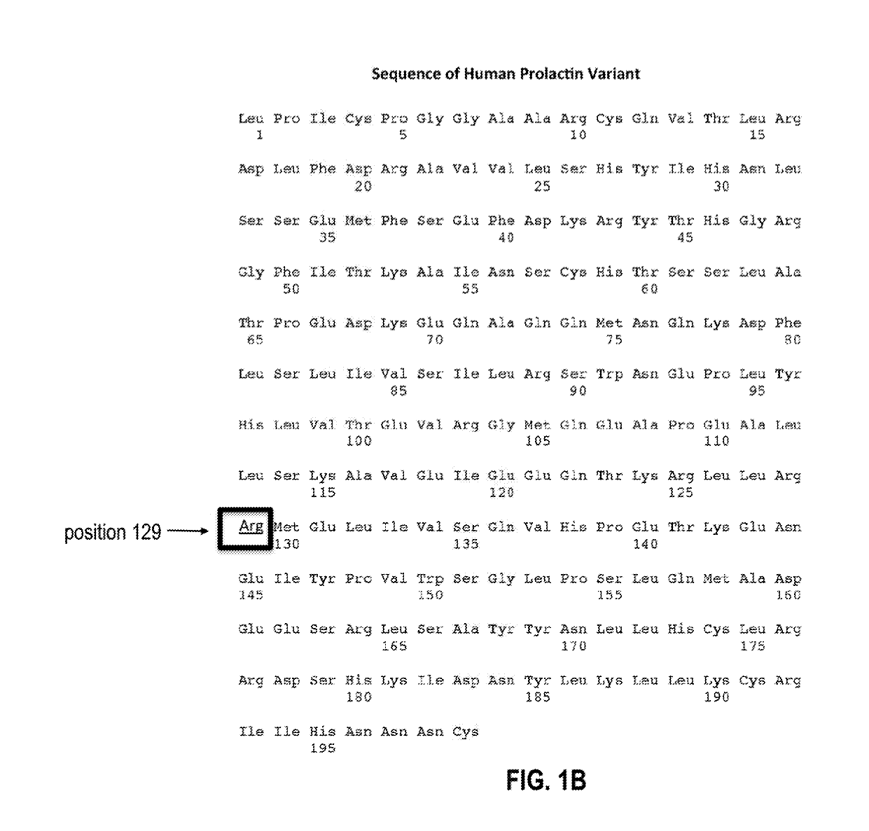

In some embodiments, PRL variants, PRLR antagonists, and PRLR inhibitors, which antagonize the action of PRL at its receptor, refer herein to human and nonhuman forms of the hormone PRL. The amino acid sequence of hPRL is shown in FIG. 1A.

In some embodiments, a PRLR antagonist is a variant of hPRL having an amino acid substitution of the glycine at position 129 with another amino acid. The glycine amino acid may be substituted with any naturally occurring or synthetic amino acid other than glycine. The substitution may be the sole variation from the native sequence or one of several alterations (e.g., insertions, deletions, and/or substitutions of amino acids). The substituent amino acid may be neutral-polar amino acids such as alanine, valine, leucine, isoleucine, phenylalanine, proline, methionine; neutral non-polar amino acids such as serine, threonine, tyrosine, cysteine, tryptophan, asparagine, glutamine, aspartic acid; acidic amino acids such as aspartic and glutamic acid; and basic amino acids such as arginine, histidine, or lysine. In some embodiments, the glycine at position 129 of hPRL may be substituted with valine, leucine, isoleucine, serine, threonine, proline, tyrosine, cysteine, methionine, arginine, histidine, tryptophan, phenylalanine, lysine, asparagine, glutamine, aspartic acid, and glutamic acid. In one embodiment, the substitution replaces the glycine at position 129 with arginine (G129R). FIG. 1B shows the amino acid sequence of hPRL variant (G129R hPRL) by substituting glycine with arginine at position 129. In some embodiments, a PRL variant is provided wherein the glycine at position 129 is deleted.

In some embodiments, a PRL suppressor includes a mutation in a receptor activation or heterodimerization domain. For example, when a residue larger than alanine is substituted for glycine at position 120 of human GH (hGH), the GH variant binds the first receptor site but binding of the second receptor site is impaired, preventing receptor activation. Further for example, a mutant form of PRL (G129R-hPRL) in which the glycine (G129) of helix 3 of hPRL is substituted with arginine (G129R) acts as a receptor antagonist. Substituting arginine for glycine at position 129 prevents a second receptor from binding the first receptor to form a functional complex. Further, a modified PRLR antagonist is Δ1-9-G129R-hPRL, in which the first 9 N-terminal residues of G129R-hPRL are removed. Δ1-9-G129R-hPRL functions as a pure antagonist of human PRLRs. In some embodiments, a PRL variant is provided wherein the glycine at position 129 is substituted with a natural or synthetic amino acid other than glycine and there is a deletion of at least the 9 N-terminal residues and up to the 14 N-terminal residues.

In one embodiment, a PRL variant is provided known as Δ1-9-G129R-hPRL, wherein the glycine at position 129 is substituted for arginine and there is a deletion of the 9 N-terminal residues.

In one embodiment, a PRL variant is provided known as Δ1-14-G129R-hPRL, wherein the glycine at position 129 is substituted for arginine and there is a deletion of the 14 N-terminal residues.

In some embodiments, the present disclosure provides for the use of antibodies specific for PRL and/or PRLR for the treatment of sexual dysfunction, including but not limited to treating prolonged refractory period in a patient.

The present disclosure also provides for the use of antibodies specific for PRL and/or PRLR alone or in combination with other PRL suppressors with or without PDE5 inhibitors to treat sexual dysfunction.

A number of antibodies specific for PRL and/or PRLR have been developed or are currently under development in order to aid in treating certain cancers. These antibodies act to reduce PRL or PRLR, and in the present disclosure, provide treatment for sexual dysfunctions.

In one embodiment, the PRLR antibody LFA102 is used to treat sexual dysfunction, including the treatment of prolonged refractory period. LFA102 is a humanized neutralizing monoclonal antibody directed against the extracellular domain of PRLR. This antibody was found to effectively antagonize PRL-induced signaling in breast cancer cells in vitro and in vivo and to block PRL-induced proliferation in numerous cell line models, including examples of autocrine/paracrine PRL activity.

In some embodiments, the PRL suppressor is conjugated to one or more chemical groups, such as polyethylene glycol (PEG), that increase the actual molecular weight of the PRL suppressor to between about 22 and about 200 kDa. In some embodiments, the molecular weight is increased to between 25-50 kDa, 50-100 kDa, 100-150 kDa, or 150-200 kDa.

In some embodiments, one or more PRL suppressors may be pegylated with PEG and used alone or in combination with a PDE5 inhibitor to treat sexual dysfunction. In some embodiments a PRL suppressor is conjugated to between about one to about six molecules of PEG. In some variations, a PRL suppressor may be conjugated to 1, 2, 3, 4, 5, 6, 7, 8, 9, or 10 PEG molecules. In some embodiments, PRL variants such as G129R-hPRL or Δ1-9-G129R-hPRL may be pegylated. Such PRL suppressors may be delivered in many ways, including but not limited to: oral delivery, transcutaneous delivery, transdermal delivery, nasal or pulmonary delivery (inhaled), rectal delivery, or by other methods of drug delivery known in the art. In some embodiments, the present disclosure provides for treatment of sexual dysfunction with one or more PRL suppressors via inhaled formulations that may or may not be pegylated alone or in combination with a PDE5 inhibitor.

In some embodiments, the present disclosure provides for treatment of sexual dysfunction with one or more PRL suppressors with sustained release, where the sustained release method includes a zinc complex, mircospheres, hydrodel, or other methods known in the art.

In some embodiments, the present disclosure provides for treatment of sexual dysfunction with one or more PRL suppressors with prolonged half-life, where the half-life is prolonged by PEGylation, albumin conjugation, XTEN amino sequence fusion, hybrid Fc fusion, carboxy-terminal peptide fusion, extracellular receptor of hGH fusion, or other methods known in the art.

In some embodiments, the present disclosure provides for treatment of sexual dysfunction with one or more PRL suppressors via intranasal delivery.

In some embodiments, the present disclosure provides for treatment of sexual dysfunction with one or more PRL suppressors with transdermal delivery, where the transdermal mechanisms include radio-frequency ablation, microneedle patch, self-dissolving micropiles, or other methods known in the art for transdermal delivery.

In one embodiment, a method of producing a pegylated PRL suppressor to treat sexual dysfunction comprises: pegylating a PRL suppressor; applying the pegylated PRL suppressor to a cation exchange chromatography column; and eluting the pegylated PRL suppressor.

In one embodiment, the PRL suppressor is conjugated to one or more chemical groups, such as PEG, in a pharmaceutical composition of a unit dosage capsule that may comprise a dry powder suitable for pulmonary administration by a patient to the deep lung. In some embodiments, the capsule contains a unit dosage of about 0.5 mg to about 50 mg of the pharmaceutical composition, or any sub-range there between. For example, in some embodiments, the unit dosage is about 1.0 mg to about 30.0 mg, about 2.5 mg to about 25 mg, about 5 mg to 30 mg, 25 mg to 50 mg, or any subrange there between. In some embodiments, the capsule contains a unit dosage of 3.0 mg, 4.8 mg, 5.0 mg, 6.0 mg, 9.0 mg, 10.0 mg, 12.0 mg, 15.0 mg, 15.1 mg, 20.0 mg, or 21.1 mg.

In some embodiments, one or more dopamine agonists are used alone or in combination with other PRL suppressors with or without PDE5 inhibitors to treat sexual dysfunction. Dopamine activity decreases the release of PRL from the pituitary gland and thus dopamine agonists reduce levels of PRL in the body when used alone or in combination with other PRL suppressors with or without combined use of PDE5 inhibitors.

Examples of dopamine agonists include, but are not limited to: Apomorphine, Aripiprazole Bromocriptine, Cabergoline, Ciladopa, Dihydrexidine, Dinapsoline, Doxanthrine, Epicriptine, Fenoldopam, Lisuride, Pergolide, Phencyclidine, Piribedil, Pramipexole, Propylnorapomorphine, Quinagolide, Quinpirole, Ropinirole, Rotigotine, Roxindole, Salvinorin A, and Sumanirole.

GH and PRL are structurally similar, and GH has the ability to bind the PRLR. In the presence of zinc, the binding affinity of hGH for the extracellular binding domain of the hPRLR is increased significantly. GH variants, such as G120R (glycine 120 replaced with arginine) hGH, also have the ability to bind PRLR. Thus, GH and GH variants may have the ability to act as competitive inhibitors to PRL at the PRLR.

In addition to PRL and GH, PL is also structurally similar and these hormones are part of the Somatotropin family. PL is also able to bind the PRLR.

Examples of members of the Somatotropin family include: PRL; GH; Choriomammotropin (lactogen); its placental analogue—PL; placental prolactin-related proteins; Proliferin and Proliferin related protein; and Somatolactin.

By binding PRL, members of the Somatotropin family can either act as agonists (activating the PRLR) or antagonists (via competitive inhibition with PRL and preventing PRLR from activating). By acting as PRLR antagonists, members of the Somatotropin family may be used to treat sexual dysfunctions, such as prolonged refractory period.

In some embodiments, sexual dysfunction may be treated using zinc. Zinc (Zn²⁺) controls more than 300 different enzymes, many of them involved in intermediary metabolism, DNA and RNA synthesis, gene expression, and immunocompetence. Zinc can inhibit PRL secretion within a range of physiologically and pharmacologically relevant concentrations.

For example, zinc supplementation for dialysis patients with high PRL levels, reduced their PRL levels and improved sexual activity (Campieri et al. “Prolactin, zinc and sexual activity in dialysis patients.” Proc Eur Dial Transplant Assoc 16 (1979): 661-2.).

Further for example, when zinc is added to GH variant, G120R, its agonistic activities are increased, and the activity of the GH receptor diminishes dramatically as compared to wild-type GH (Duda and Brooks. “Differential effects of zinc on functionally distinct human growth hormone mutations.” Protein Engineering 16, 7 (2003): 531-534.).

Due to the dramatic effect of zinc on hGH receptor activity with G120R hGH, zinc in combination with some PRL suppressors, may have a synergistic effect on reducing PRL secretion and/or PRLR activity.

Thus, zinc in combination with other PRL suppressors such as PRL variants, G129R-hPRL or Δ1-9-G129R-hPRL, with and without PDE5 inhibitors may be used to treat sexual dysfunction, such as male refractory period.

In some embodiments, GH or GH variants, alone or in combination with zinc and/or with other PRL suppressors and/or with PDE5 inhibitors are used for the treatment of sexual dysfunction, including but not limited to treating prolonged refractory period in a patient.

In some embodiments, PL, lactogen variants, truncated lactogen receptor, and other proteins or variants of the Somatotropin family, alone or in combination with zinc and/or with other PRL suppressors and/or with PDE5 inhibitors may be used for the treatment of sexual dysfunction, including but not limited to treating prolonged refractory period.

In the treatment of sexual dysfunction, the PRL suppressor may be administered either in isolation or as part of a sequential or combined treatment regimen. For example, PRL suppressors such as a PRL variant or PRL-BP may be administered in a combined regimen with one or more PDE5 inhibitors and/or zinc.

Described herein are compositions comprising a PRL suppressor in a suitable pharmaceutical carrier. In some embodiments, one or more PRL suppressors may be delivered to a patient in a number of ways including, but not limited to: oral delivery, transcutaneous delivery, transdermal delivery, nasal or pulmonary delivery (inhaled), 685 rectal delivery, or by other methods of drug delivery known in the art. In one embodiment, the PRL suppressor is delivered via an inhalable powder as described elsewhere herein. In one embodiment, the PRL suppressor is delivered via microneedle described elsewhere herein.

Pharmaceutical compositions suitable for use include compositions comprising a PRL suppressor (e.g., a PRL variant or PRL-BP) in an effective amount to achieve its intended purpose. More specifically, an effective dose refers to the amount of PRL suppressor required to inhibit actions of the PRLR thereby decreasing the symptoms associated with a sexual dysfunction, such as lengthened refractory period.

The amount of the composition will, of course, also be dependent on the subject being treated, the sexual dysfunction being treated, the severity of the symptoms of the disorder, and the judgment of the prescribing physician. In some embodiments, it may be necessary to adjust the treatment to a lower or higher dose if the clinical response is not adequate. In some embodiments, an inhalable powder formulation comprising a PRL suppressor (such as a prolactin variant like G129R-hPRL or Δ1-9-G129R-hPRL) as an active ingredient for treating one or more conditions of sexual dysfunction (such as prolonged refractory period) is used.

In some embodiments, a microneedle patch delivering PRL suppressor (e.g., G129R-hPRL, Δ1-9-G129R-hPRL) as an active ingredient for treating one or more conditions of sexual dysfunction (e.g., prolonged refractory period) is used.

In some embodiments, a method for preventing or treating sexual dysfunction (such as prolonged refractory period) in a subject includes administering a therapeutically effective amount of a PRL suppressor via a microneedle patch.

In some embodiments, a microneedle patch for preventing or treating a sexual dysfunction related condition comprises a PRL suppressor (such as a PRL variant like G129R-hPRL or Δ1-9-G129R-hPRL) as an active ingredient.

In some embodiments, a method for preventing or treating sexual dysfunction (such as prolonged refractory period) in a subject includes administering a therapeutically effective amount of an inhalable powder formulation comprising a PRL suppressor.

In some embodiments, an inhalable powder formulation for preventing or treating a sexual dysfunction related condition comprises a PRL suppressor (such as a PRL variant like G129R-hPRL or Δ1-9-G129R-hPRL) as an active ingredient.

The term “powder” or “powdered” refers to a formulation that consists of finely dispersed solid particles that are relatively free flowing and capable of being dispersed in an inhalation device and subsequently inhaled by a subject so that the particles reach the lungs to permit penetration into the alveoli.

The term “dispersibility” refers to the degree to which a powder formulation can be dispersed (i.e., suspended) in a current of air so that the dispersed particles can be respired or inhaled into the lungs of a subject. For example, a powder formulation that is only 10% dispersible means that only 10% of the mass of finely-divided particles making up the formulation can be suspended for oral inhalation into the lungs; 50% dispersibility means that 50% of the mass can be suspended.

The term “therapeutically effective amount” is the amount of PRL suppressor present in the powder formulation that is needed to provide the desired level of the PRL suppressor to a subject to be treated to give the anticipated therapeutic response.

The term “pharmaceutically acceptable” refers to an excipient, a carrier or other additives used in the formulation that can be taken into the lungs with no significant adverse toxicological effects on the lungs.

The PRL suppressor used in the formulation may be any of the PRL suppressors described elsewhere herein including: PRLR antagonists, PRLR inhibitors, PRL variants, truncated PRLR, serum PRL-BP, soluble isoforms of the PRLR, dopamine agonists, PRL antibodies, PRLR antibodies, GH, GH variants, PL, PL variants, truncated lactogen receptors, and other proteins or variants of the somatotropin family, zinc, estrogen, estrogen variants, progesterone, and progesterone variants. In one embodiment, the PRL suppressor is a PRL variant such as G129R-hPRL or Δ1-9-G29R-hPRL.

The inhalable powder formulation may comprise a suitable pharmaceutically-acceptable excipient in addition to a therapeutically effective amount of PRL suppressor. Such excipients must be physiologically acceptable when used in administration by the aerial pathways.

The excipients which satisfy this requirement will be selected from a group comprising: monosaccharides such as glucose and arabinose; disaccharides such as lactose, saccharose and maltose; polysaccharides such as sorbitol, mannitol and xylitol; salts such as sodium chloride, calcium carbonate, and sodium phosphate; sodium bicarbonate; amino acids; peptides; polymers; lipids; and a mixture thereof.

In some embodiments, the excipients are mono-, di- or polysaccharides. In one embodiment, the excipient is lactose.

In some embodiments, the excipients are salts. In one embodiment, the excipient is sodium phosphate.

In some embodiments, surfactants, such as dipalmitoyl phosphatidylcholine (DPPC) may be incorporated to the formulation to further improve powder flow, aerosol dispersion, and lung deposition.

In some embodiments, a particulate active ingredient suitable for inhalation therapy may be incorporated into the formulation such as a corticosteroid (e.g., fluticasone propionate) or a bronchodilator (e.g., salmeterol or albuterol or a salt thereof).

It will be appreciated that the formulations described herein may contain minor amounts of other additives or coatings, for example, taste masking agents or sweeteners, delayed release or activity compositions, additives for improving absorption and/or ingestion, or any other additive or coating relevant for the indication and/or delivery route.

In some embodiments, the formulation may be formulated so as to provide rapid, sustained, or delayed release of the active ingredient after administration to a patient by employing any of the procedures well known in the art.

In one embodiment, an inhalable powder formulation may include a PRL variant such as G129R-hPRL or Δ1-9-G129R-bPRL, lactose, and dipalmitoyl phosphatidylcholine (DPPC).

In one embodiment, an inhalable powder formulation may include a PRL suppressor (such as G129R-hPRL or Δ1-9-G129R-hPRL), lactose and dipalmitoyl phosphatidylcholine (DPPC) in the ratio of 1:0.5 to 1.5:1.5 by weight, preferably 1:1:3 by weight.

In one embodiment, an inhalable powder formulation may include a PRL variant such as G129R-hPRL or Δ1-9-G129R-hPRL, sodium phosphate and dipalmitoyl phosphatidylcholine (DPPC).

The inhalable powder formulation may be prepared by spray drying process as described in the art (Bosquillon et al. “Pulmonary delivery of growth hormone using dry powders and visualization of its local fate in rates.” J Control Rel 96 (2004): 233-244.).

For example, the inhalable powder formulation comprising PRL suppressor, DPPC and lactose may be prepared by a method comprising: combining DPPC dissolved in a solvent (e.g., ethanol) with an aqueous solution comprising PRL suppressor and lactose to obtain an aqueous mixture; and subjecting the aqueous mixture to a spray drying process to obtain an inhalable powder formulation.

The inhalable powder formulation can be advantageously delivered by dry powder inhaler or by metered dose inhaler. For delivery of dry inhalable powder, the PRL suppressor (such as G129R-hPRL or Δ1-9-G129R-hPRL) is milled, precipitated, spray dried, or otherwise processed to particle sizes (mass median aerodynamic diameter; MMAD) between about 1 and 10 μm. In one embodiment, the particle size is between about 1 and 5 μm.

The dry powder formulation is practical and convenient for ambulatory use because it does not require dilution or other handling, it has an extended shelf-life and storage stability, and the dry powder inhalation delivery devices are portable and do not require an air compressor needed by aerosol nebulizers.

All techniques suitable for preparation of dry inhalable powders and any and all improvements thereof as well as any dry powder inhaler are intended to be within the scope of the present disclosure.

The inhalable formulation comprising a PRL suppressor (such as G129R-hPRL or Δ1-9-G129R-hPRL) is efficaciously delivered to a patient's endobronchial space of airways by inhalation of a dry powder.

The formulation may be delivered from a unit dosage receptacle comprising an amount that will be sufficient to provide the desired physiological effect upon inhalation by a subject in need thereof. The amount may be dispersed in a chamber (or insufflator) that has an internal volume sufficient to capture substantially all of the powder dispersion resulting from the unit dosage receptacle.

The effective daily dose of the PRL suppressor for the treatment of sexual dysfunction conditions can be administered in a single dose or in divided doses. However, it should be understood that the amount of the active ingredient actually administered ought to be determined in light of various relevant factors including the condition to be treated, the age and weight of the individual patient, the severity of the patient's symptom, and other relevant factors.

The inhalation of PRL suppressor (such as G129R-hPRL or Δ1-9-G129R-hPRL) permits an administration of small yet effective amount of PRL suppressor directly into lungs.

The inhalable PRL suppressor (e.g., G129R-hPRL or Δ1-9-G129R-hPRL) may be an efficacious, safe, nonirritating and physiologically compatible formulation suitable for the treatment of sexual dysfunction conditions.

In addition, an inhalable powder formulation for preventing or treating a sexual dysfunction condition may comprise a PRL suppressor as an active ingredient.

In one embodiment, Δ1-9-G129R-hPRL is buffered with sodium phosphate and administered through pulmonary delivery into a patient's systemic circulation prior to orgasm. Δ1-9-G129R-hPRL binds to PRLR, acting as a PRLR antagonist and acts in competitive inhibition of PRL that is released with orgasm. By binding PRLR, Δ1-9-G129R-hPRL prevents PRL from exerting effects on PRLR that lead to the sexual refractory period in men.

In some embodiments, the particle consists of about 93.5% PRL suppressor (such as G129R-hPRL or Δ1-9-G29R-hPRL) and about 6.5% sodium phosphate. In some embodiments, the particle consists of about 85% to about 99% PRL suppressor (or any sub-range there between) and about 15% to about 1% sodium phosphate (or any sub-range there between). In some embodiments, the particle comprises 1%, 2% 3%, 4%, 5%, 6%, 7%, 8%, 9%, 10%, 11%, 12%, 13%, 14%, or 15% sodium phosphate. In some embodiments, the particle comprises 0.5%, 1.5%, 2.5%, 3.5%, 4.5%, 5.5%, 6.5%, 7.5%, 8.5%, 9.5%, 10.5%, 11.5%, 12.5%, 13.5%, 14.5%, or 15.5% sodium phosphate. In some embodiments, the particle comprises 85%, 86%, 87%, 88%, 89%, 90%, 91%, 92%, 93%, 94%, 95%, 96%, 97%, 98%, 99%, or 100% PRL suppressor. In some embodiments, the particle comprises 85.5%, 86.5%, 87.5%, 88.5%, 89.5%, 90.5%, 91.5%, 92.5%, 93.5%, 94.5%, 95.5%, 96.5%, 97.5%, 98.5%, or 99.5% PRL suppressor.

### Prophetic Example 1

The following prophetic example serves to provide approximate dosage levels of PRL suppressors to achieve the intended effect, for example treatment of male refractory period. Based on the literature, a few assumptions about dosage can be made, as will be described in further detail below.

After male orgasm, PRL levels range from 7.5 ng/mL to 15 ng/mL (Haake et al. “Absence of orgasm-induced prolactin secretion in a healthy multi-orgasmic male subject.” Int J impot Res 14 (2002): 122-135.). After orgasm, male and female PRL levels are 5-12 ng/mL and 10-60 ng/mL, respectively (Exton et al. “Coitus-induced orgasm stimulates prolactin secretion in healthy subjects.” Psychoneuroendocrinology 26 (2001): 287-294.).

The PRL suppressor, G129R-hPRL, has 10 fold lower binding affinity compared to hPRL (human PRL) for the PRLR (Goffin et al. “Drug Insight: prolactin-receptor antagonists, a novel approach to treatment of unresolved systemic and local hyperprolactinemia?” Nat Clin Practice Endocrinol Metab 2 (2006): 571-581.). Further, efficient competitive antagonism of wild-type hPRL requires at least a 10-50-fold molar excess of the PRL suppressor, Δ1-9-G129R-hPRL (Goffin et al. “Drug Insight: prolactin-receptor antagonists, a novel approach to treatment of unresolved systemic and local hyperprolactinemia?” Nat Clin Practice Endocrinol Metab 2 (2006): 571-581.).

Based on these data, it is deduced that for very 1 mol of hPRL, at least 10 mols of G129R-hPRL are needed, or 10-50 mols of Δ1-9-G129R-hPRL are needed. Thus to counteract 5 to 15 ng/ml of PRL (male orgasm), a 10-fold greater dose of PRL suppressor (e.g., G129R-hPR or Δ1-9-G129R-hPRL) is needed. Therefore, for counteracting 5 to 15 ng/mL PRL, 50 to 750 ng/mL of PRL suppressor (e.g., G129R-hPRL or Δ1-9-G129R-hPRL) may be needed.

In some embodiments, a 2-fold greater dose of PRL suppressor may be needed, for example 10 to 30 ng/mL. In some embodiments, a 5-fold greater dose of PRL suppressor may be needed, for example 25 to 75 ng/mL. In some embodiments, a 20-fold greater dose of PRL suppressor may be needed, for example 100 to 300 ng/mL. In some embodiments, a 25-fold greater dose of PRL suppressor may be needed, for example 125 to 375 ng/mL. In some embodiments, a 50-fold greater dose of PRL suppressor may be needed, for example 250 to 750 ng/ml.

In some embodiments, to treat male refractory period, a range of 10-20, 20-30, 30-40, 40-50, 50-100, 100-150, 150-200, 200-250, 250-300, 300-350, 350-400, 400-450, 500-550, 550-600, 600-650, 650-700, or 700-750 ng/mL of PRL suppressor (e.g., G129R-hPRL or Δ1-9-G129R-hPRL) may be needed in the plasma before natural PRL is released with orgasm to prevent male refractory period

In some embodiments, bioavailability of PRL suppressor may vary. In some such embodiments, an increased amount of PRL suppressor may need to be delivered to the body to achieve the desired therapeutic effect. For example, the bioavailability of GH from inhaled GH trials is an average of 3.5% (range of 2.7% to 4.4%). Assuming inhaled PRL suppressor (e.g., G129R-hPRL) has a similar bioavailability as GH (e.g., 3.5%), then about 1,429 to 21,429 ng/mL may need to be delivered via an inhaler. In some embodiments, based on bioavailability, 285-572; 572-858; 858-1,143; 1,143-1,429; 1,429-2,857; 2,857-4,286; 4,286-5,714; 5,714-7,143; 7,143-8,571-10,000; 10,000-11,428; 11,428-12,857; 12,857-14,285; 14,285-15,714; 15,714-17,142; 17,142-18,571; 18,571-20,000; or 20,000-21,429 ng/mL may need to be delivered by an inhaler.

Depending on inhaled technology, oral peptide technology or microneedle technology, the dosage delivered may vary.

However, regardless of delivery system, PRL suppressor should be active in the plasma prior to orgasm to counteract 5 to 15 ng/mL of hPRL during male orgasm. For PRL suppressors G129R-hPRL or Δ1-9-G129R-hPRL, about or substantially 50 to 750 ng/mL is needed to be active in the plasma prior to orgasm to counteract 5 to 15 ng/mL of hPRL during male orgasm.

Further, depending on the PRL suppressor's half-life and/or sustained release status, the bioavailability of the PRL suppressor may vary, affecting the dose required to inhibit or outcompete hPRL. Human PRL has a half-life of 40.8+/−13.8 min as described by Yoshida and colleagues (Yoshida et al. “A kinetic study on serum prolactin concentration in the thyrotropin-releasing hormone test” Kaku Igak 6 (1991): 585-590). Others describe the in-vivo plasma half-life of wild type human PRL as 15-20 minutes (Bernichtein et al. “Development of Pure Prolactin Receptor Antagonists” Biol Chem 38 (2003): 35988-99). G129R-hPRL injection used in experiments by Chen and colleagues (U.S. Pat. No. 8,754,031 B2) report a half-life of 1-2 hours. Cai and colleagues (Cai et. al, “Developments in human growth hormone preparations: sustained-release, prolonged half-life, novel injection devices, and alternative delivery routes” Int J Nanomedicine 9 (2014): 3527-3538) describe half-life of subcutaneous hGH as 3.6 hrs vs intravenous half-life of 0.36 hrs.

There are different means to change the half-life of proteins and a commonly used procedure is to PEGylate the protein of interest. An alternative it is create conjugates to albumin or to fuse the protein of interest to the Fc portion of antibodies. In the case of PRL suppressors for treatment of sexual dysfunction, the need to change half-life will depend on the route of administration. Regardless of the half-life or sustained release of the PRL suppressor, the active PRL suppressor delivered to the plasma must counteract 5 to 15 ng/mL of hPRL released during male orgasm.

In some embodiments, the frequency of dosing is one dose prior to sexual activity. In some embodiments, the frequency of dosing is one dose prior to sexual activity and one dose after orgasm. In some embodiments, one or more doses may be required to achieve the desired therapeutic effect, for example 12 hours, 8 hours, 4 hours, 2 hours, or 1 hour before sexual activity (e.g., coitus) or 10 minutes, 15 minutes, 20 minutes, 30 minutes, 45 minutes, or 60 minutes after sexual activity (e.g., coitus).

In some embodiments, the following steps would be taken to determine the effective dose to treat sexual dysfunction, for example male refractory period. The amount of prolactin released by one or more individuals at orgasm is measured. Based on the range of the measured amount, the amount of prolactin suppressor needed to suppress the released amount of prolactin is calculated. Depending on the delivery system chosen and associated half-life, sustained release (e.g., embed PRL suppressor in insoluble porous matrix, matrix that swells to form a gel through which PRL suppressor exits, osmotic controlled-release oral delivery system, etc.), and bioavailability, the effective dose of prolactin suppressor can be calculated.

In preformulations of the PRL suppressor, the physical, chemical, and/or mechanical properties of the PRL suppressor may be characterized to understand how the PRL suppressor will behave under stress conditions (e.g., freeze/thaw cycles, temperature fluctuations, shear stress, etc.) and to determine suitable excipients in the formulation, for example sodium phosphate or lactose.

In determining a suitable formulation for administration of a PRL suppressor to an individual, the following properties, which affect bioavailability, of the PRL suppressor may be determined: particle size, pH, solubility, and/or polymorphisms. The formulation may have an acceptable taste and/or dispersion.

### Prophetic Example 2

The following prophetic example serves to provide examples of techniques available to clone, synthesize, and/or generate DNA (e.g., cDNA) and/or RNA encoding PRL suppressors and/or protein PRL suppressors.

The PRL variants may be prepared by chemical synthesis or by recombinant DNA techniques. Generally, a cDNA of PRL may be prepared using standard PCR amplification techniques, RNA or cDNA prepared from a cell which produces PRL (such as a pituitary cell) as a template, and oligonucleotide primers designed based on known PRL nucleic acid or amino acid sequence. A nonlimiting example of the preparation of a cDNA encoding hPRL is set forth in Example 7 of European patent EP2316467A1, filed May 11, 1999, the disclosure of which is incorporated herein by reference in its entirety.

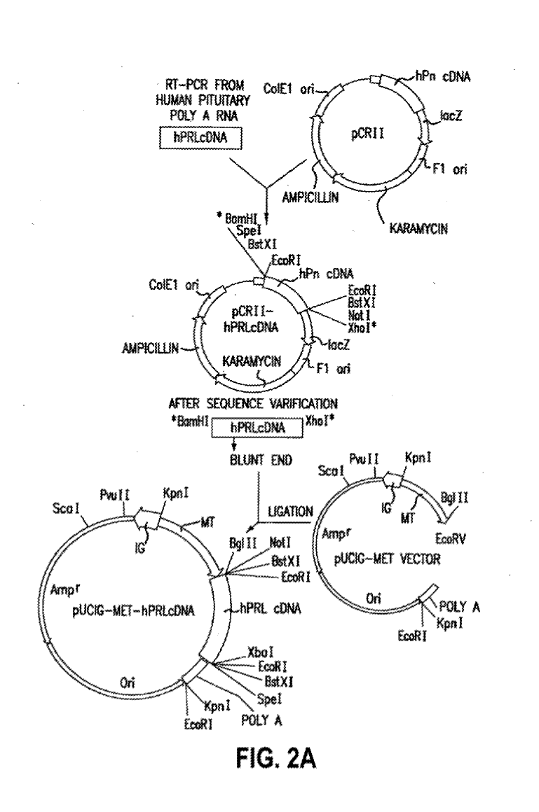

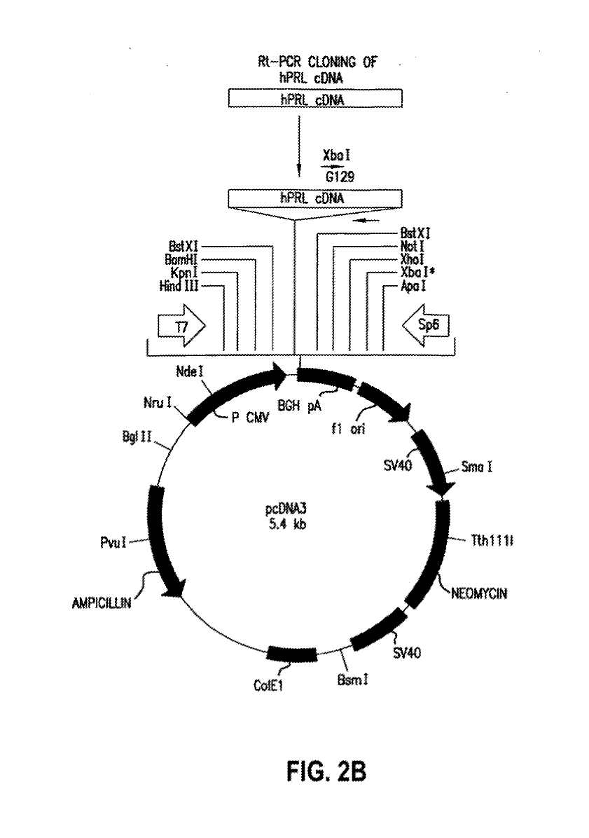

FIG. 2A illustrates a schematic representation of the cloning and construction of the expression plasmid pUCIG-MT-hPRL-cDNA.

FIG. 2B illustrates the plasmid map and general strategy of PCR-directed mutagenesis. pcDNA3, the parental vector, contains human immediate-early cytomegalovirus (CMV) transcriptional regulatory sequences and a polyadenylation signal and transcription termination sequence from bovine GH gene (BGH pA). hPRL cDNA can be cloned using RT-PCR from human pituitary mRNA and inserted into BstX1 sites. Mutation is generated by designing PCR primers at Xba I sites.

Alterations are introduced into the PRL cDNA either randomly or by directed mutagenesis. An example of the use of oligonucleotide mediated site-directed mutagenesis is also set forth in Example 7 of European patent EP2316467A1, filed May 11, 1999, the disclosure of which is incorporated herein by reference in its entirety, and illustrates the introduction of the G129R substitution into hPRL.

Where the PRL variant is to be produced by recombinant techniques, a nucleic acid encoding the PRL variant may be incorporated into an expression vector, operatively linked to a suitable promoter/enhancer sequence. The expression vector may further contain one or more elements which aid in the expression of the PRL variant, including a transcription termination site, a polyadenylation site, a ribosome binding site, a signal sequence, etc. Suitable expression systems include mammalian cells, insect cells, plant cells, yeast cells, slime mold, and organisms, including transgenic plants and transgenic animals. Suitable expression vectors include herpes simplex viral based vectors such as pHSV1 (Geller et al. “An efficient deletion mutant packaging system for defective herpes simplex virus vectors: potential applications for human gene therapy and neuronal physiology.” Proc Natl Acad Sci USA 87 (1990): 8950-8954.); retroviral vectors such as MFG (Jaffee et al. “High efficiency gene transfer into primary human tumor explants without cell selection.” Cancer Res 53 (1993): 2221-2226.), and in particular Moloney retroviral vectors such as LN, LNSX, LNCX, LXSN (Miller and Rosman. “Improved retroviral vectors for gene transfer and expression.” Biotechniques 7 (1989): 980-989.); vaccinia viral vectors such as MVA (Sutter and Moss. “Nonreplicating vaccinia vector efficiently expresses recombinant genes.” Proc Natl Acad Sci USA 89 (1992): 10847-10851.); adenovirus vectors such as pJM 17 (Ali et al. “The use of DNA viruses as vectors for gene therapy.” Gene Therapy 1 (1994): 367-384.; Berkner. “Development of adenovirus vectors for the expression of heterologous genes.” Biotechniques 6 (1988): 616-624.; Wang and Finer. “Second-geeration adenovirus vectors.” Nature Medicine 2 (1996): 714-716.); adeno-associated virus vectors such as AAV/neo(Muro-Cacho et al. “Gene transfer in human lymphocytes using a vector based on adeno-associated virus.” J Immunother 11 (1992): 231-237.); lentivirus vectors (Zufferey et al. “Multiply attenuated lentiviral vector achieve efficient gene delivery in vivo.” Nature Biotechnology 15 (1997): 871-875.); plasmid vectors such as pCDNA3 and pCDNA1 (InVitrogen), pET 11a, pET3a, pET11d, pET3d, pET22d, and pET12a (Novagen); plasmid AH5 (e.g., contains the SV40 origin and the adenovirus major late promoter), pRC/CMV (InVitrogen), pCMU II, pZipNeo SV (Paabo et al. “Structural and functional dissection of an MHC class I antigen-binding adenovirus glycoprotein.” EMBO J 5 (1986): 1921-1927.), pSR□□ (DNAX, Palo Alto, Calif.) and pBK-CMV; and baculovirus expression vectors (O'Reilly et al. (1995) “Baculovirus Expression Vectors.” Oxford University Press.) such as p2Bac (InVitrogen).

A PRL variant produced in a recombinant expression system may then be purified by standard techniques, including electrophoresis, chromatography (including affinity chromatography), and ultrafiltration.

### Prophetic Example 3

The following prophetic example serves to provide example techniques for generating, isolating, and/or purifying DNA, RNA, and/or protein PRL suppressors.

The present disclosure provides for truncated PRLR, serum PRL binding proteins, and soluble isoforms of the PRLR (collectively referred to herein as PRL-BP(s)), which retain the ability to bind to PRL and therefore are able to compete with the cell surface forms of PRLR for PRL binding, thereby inhibiting the ability of PRL to interact with its receptor. The present disclosure provides for the use and method of treating sexual dysfunction conditions, such as prolonged refractory period with PRL suppressors, for example PRL-BP(s).

A PRL-BP may be prepared by removing all or a part of the transmembrane and/or intracellular domains of the PRLR, either enzymatically or using recombinant DNA techniques. In one embodiment, the PRLR to be truncated is as described in Boutin et al. (Boutin et al. “Identification of a cDNA encoding a long form of prolactin receptor in human hepatoma and breast cancer cells.” Mol Endocrinol 3 (1989): 1455-1461.), the disclosure of which is herein incorporated by reference in its entirety.

For recombinant preparation, nucleic acid molecules encoding the native PRLR may be prepared and then altered to encode a PRL-BP. For example, but not by way of limitation, the PRLR may be cloned using techniques as set forth in Example 9 of European patent EP2316467A1, filed May 11, 1999, the disclosure of which is incorporated herein in its entirety.

The amino acid sequence of PRLR from a variety of different organisms is known. The human PRLR sequence is obtainable from Genbank Accession No: 13032. Further, the amino acid residues which delineate the extracellular, transmembrane, and cytoplasmic domains of the PRLR are also known (Kelly et al. “Purification, cloning, and expression of the prolactin receptor.” Biol Reprod 40 (1989): 27-32.). Given the elucidation of these domains, one skilled in the art would readily be capable of producing a truncated form of PRLR which retains the ability to bind PRL, but which may by used to inhibit the effects of PRL.

Recombinant DNA methods which are well known to those skilled in the art can be used to construct expression vectors containing PRL-BP coding sequences and appropriate transcriptional/translational control signals. The efficiency of expression can be enhanced by the inclusion of appropriate transcriptional enhancer elements, transcriptional terminators, etc. The methods may include in vitro recombinant DNA and synthetic techniques and in vivo recombinants (See, for example, Sambrook et al. (1989) “Molecular Cloning: A Laboratory Manual.” Cold Spring Harbor Press, Cold Spring Harbor, N.Y., and Glover, D. M., (ed.) (1985) “DNA Cloning: A Practical Approach.” MRL Press, LTD., Oxford, U.K., vol. I and II), both of which are incorporated by reference herein in their entirety.

When recombinant DNA technology is used to produce PRL-BP, it may be advantageous to engineer fusion proteins that can facilitate, for example, solubility or purification. Such fusion proteins can be made by ligating the appropriate nucleic acid sequences encoding the desired amino acid sequences to each other by methods known in the an, in the proper reading frame, and expressing the fusion protein by methods commonly known in the art. The PRL-BP gene product contained within such fusion proteins may include, for example, one or more of the extracellular domains or portions (e.g., ligand-binding portion or domain).

In one embodiment, for hPRL-BP expression, a mammalian expression vector such as pcDNA3.1/His Xpress (Invitrogen Corp., San Diego, Calif.) may be used. This vector contains a human immediate-early cytomegalovirus promoter and bGH poly A addition signal. In addition, it offers an in frame (His)6 peptide at the N-terminus which allows an easy detection after purification of hPRL-BP. Recombinant hPRL-BP produced using such a vector in cell culture may be concentrated by ultrafiltration. The concentration of hPRL-BP following ultrafiltration may be determined by protein assay and confirmed by Western Blot analysis using anti-His antibody (Santa Cruse, Calif.) and may be quantified by densitometric methods (Fernadez and Kopchick. “Quantitative determination of growth hormone by immunoblotting.” Anal Biochem 191 (1990): 268-271.).

In one embodiment, a truncated PRL-BP may be made by protein synthesis techniques, e.g., by use of a peptide synthesizer. In addition, truncated PRL-BP may be prepared by purification of full length PRLR protein, from either naturally occurring or genetically engineered PRLR producing cells, followed by enzymatic cleavage of the purified protein using proteolytic enzymes, such as trypsin, to form PRL-BP.

The primary transcript of the PRLR has alternative splice products that yield different lengths of the cytoplasmic tails, each of which are reported to have distinctive signaling properties

Autocrine/paracrine regulation of the bioavailability of extrapituitary PRL for binding to its receptor has been proposed to be regulated by soluble forms of the PRLR.

These soluble receptor “binding proteins” are generated either by alternate splicing of mRNA for the receptor or through post-translational proteolytic cleavage of the extracellular domain of the receptor.

To date, multiple PRL-BPs have been described, including some that are found in human serum, milk, and cell lysates.

A 32-kDa human PRL binding protein (hPRL-BP), generated by alternative splicing or proteolysis, which is capable of binding both hPRL and hGH has been identified (Kline et al. “Identification and Characterization of the Prolactin-binding Protein in Human Serum and Milk.” J Biol Chem 276 (2001): 24760-24766.). A measurable fraction of circulating PRL (36%) was associated with the hPRL-BP, demonstrating that hPRL-BP antagonizes PRL action.

A second soluble form of the PRL receptor was subsequently described (Trott, et al. “Alternative splicing to exon 11 of human prolactin receptor gene results in multiple isoforms including a secreted prolactin-binding protein.” J Mol Endocrinol 30 (2003): 31-47.). This PRLR isoform results from alternative splicing of the mRNA from exon 7 to exon 11, thus giving its name Δ7/11. Δ7/11 is a soluble secreted form of the PRLR that directly binds to PRL. Unlike previously reported for hPRL-BP, Δ7/11 was found to be glycosylated. Lastly, the inability to detect Δ7/11 in human serum samples suggests that Δ7/11 is a tissue-specific factor responsible for local regulation of PRL function. These data highlight the role of Δ7/11 as a novel regulatory factor of PRL bioavailability.

The present disclosure provides for the use and method of hPRL-BP(s), including the 32-kDa hPRLBP, Δ7/11, and other forms of PRL receptor isoforms and PRL-BPs to be used as treatment for sexual dysfunction conditions, including prolonged refractory period.

### Prophetic Example 4

Based on the foregoing prophetic examples, an exemplary, non-limiting embodiment is described in detail below. As described herein, a user may include a male between the ages of 40 and 100, a male with an elongated refractory period between twenty minutes and two days, or a male otherwise experiencing erectile dysfunction. A desired effect of a PRL suppressor (e.g., G129R variant) for a user may include decreased refractory period, improved ability to achieve subsequent erections after the first erection, improved orgasm, and/or otherwise decreased erectile dysfunction.

The user may inhale a PRL suppressor immediately before sexual activity, one to two hours before sexual activity, immediately after sexual activity, or any time before or after sexual activity to achieve the desired effect. The PRL suppressor may be delivered to the user by a metered dose inhaler. In one non-limiting example, the powder formulation in the metered dose inhaler may include up to 21,500 ng/mL of a G129R variant of a PRL suppressor and sodium phosphate as a pharmaceutically acceptable excipient. In one non-limiting embodiment, the PRL suppressor may comprise about 93.5% of the dosage and sodium phosphate or other suitable excipient, filler, or inactive ingredient may comprise about 6.5% of the dosage. In another non-limiting embodiment, the formulation may comprise 5-10% PRL suppressor; 80% fillers, disintegrants, lubricants, glidants, and/or binders; and 10-15% compounds for improving disintegration, disaggregation, and/or dissolution.

Based on studies with inhaled human GH, a few assumptions can be made. For example, upon inhalation of the PRL suppressor formulation, the PRL suppressor (e.g., G129R variant) may reach peak serum levels within one to four hours. In one non-limiting embodiment, the PRL suppressor may reach peak serum levels within one to two hours. Observable effects of PRL inhibition or suppression (e.g., decreased refractory period) may be observable within fifteen minutes to four hours of inhalation of the PRL suppressor formulation. A half-life of a PRL suppressor (e.g., G129R variant) may be thirty to sixty minutes without pegylation and greater than four hours with pegylation or other half-life improving methods.

As used in the description and claims, the singular form “a”, “an” and “the” include both singular and plural references unless the context clearly dictates otherwise. For example, the term “prolactin suppressor” may include, and is contemplated to include, a plurality of prolactin suppressors. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the compositions and methods include the recited elements, and may additionally include any other elements. “Consisting essentially of” shall mean that the compositions and methods include the recited elements and exclude other elements of essential significance to the combination for the stated purpose. Thus, a composition or method consisting essentially of the elements as defined herein would not exclude other materials, features, or steps that do not materially affect the basic and novel characteristic(s) of the claimed invention. “Consisting of” shall mean that the compositions and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A pharmaceutical composition for the treatment of sexual dysfunction in an individual, the pharmaceutical composition comprising a prolactin variant having a glycine residue at position 129 substituted with an amino acid other than glycine.

2. The pharmaceutical composition of claim 1, wherein the amino acid is arginine.

3. The pharmaceutical composition of claim 1, wherein the prolactin variant further comprises an N-terminal deletion.

4. The pharmaceutical composition of claim 1, wherein the prolactin variant is conjugated to a H(OCH₂CH₂)ₙOH molecule, where n equals any number from one to six.

5. The pharmaceutical composition of claim 1, wherein the pharmaceutical composition is delivered to the individual by a microneedle patch.

6. The pharmaceutical composition of claim 1, wherein administration of the prolactin variant effectively reduces an effect of 5 to 15 ng/mL of prolactin in the plasma of the individual.

7. The pharmaceutical composition of claim 1, wherein the pharmaceutical composition is an inhalable composition.

8. The pharmaceutical composition of claim 7, wherein the inhalable composition has a concentration between 1,400 to 21,500 ng/mL of the prolactin variant.

9. The pharmaceutical composition of claim 7, wherein the inhalable composition is a powder composition.

10. The pharmaceutical composition of claim 7, further comprising one or more of lactose and sodium phosphate as a pharmaceutically acceptable excipient.

11. The pharmaceutical composition of claim 1, wherein the sexual dysfunction comprises one or more of the following: erectile dysfunction, prolonged sexual refractory period, premature ejaculation, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, drowsiness with orgasm, and drowsiness with sexual activity.

12. A pharmaceutical composition for the treatment of sexual dysfunction in an individual, the pharmaceutical composition comprising a prolactin suppressor.

13. The pharmaceutical composition of claim 12, wherein the prolactin suppressor is a prolactin variant in which a glycine residue at position 129 is substituted with arginine.

14. The pharmaceutical composition of claim 12, wherein the prolactin suppressor is conjugated to a H(OCH₂CH₂)ₙOH molecule, where n equals any number from 1 to 6.

15. The pharmaceutical composition of claim 12, wherein the pharmaceutical composition further comprises one or more of zinc or a PDE5 inhibitor.

16. The pharmaceutical composition of claim 12, wherein the sexual dysfunction comprises one or more of the following: erectile dysfunction, prolonged sexual refractory period, premature ejaculation, anorgasmia, decreased frequency or duration of orgasm, decreased sexual desire or libido, hypoactive sexual desire disorder, reduced penile erections, delayed ejaculation, sleepiness with orgasm or sexual activity, fatigue with orgasm or sexual activity, drowsiness with orgasm, and drowsiness with sexual activity.

17. The pharmaceutical composition of claim 12, wherein the prolactin suppressor comprises one or more of the following: prolactin variants, prolactin receptor antagonists, truncated prolactin receptors, prolactin receptor inhibitors, serum prolactin binding proteins, soluble isoforms of the prolactin receptor, zinc, dopamine agonists, prolactin antibodies, prolactin receptor antibodies, growth hormone, growth hormone variants, truncated growth hormone receptors, placental lactogen, placental lactogen variants, truncated lactogen receptors, choriomammotropin, proliferin, somatolactin, estrogen, estrogen variants, progesterone, and progesterone variants.

18. The pharmaceutical composition of claim 12, wherein the prolactin suppressor directly or indirectly binds a prolactin receptor.

19. The pharmaceutical composition of claim 12, wherein the prolactin suppressor is a soluble form of a prolactin receptor isoform that results from alternative splicing of a prolactin receptor primary mRNA transcript from exon 7 to exon 11.

20. The pharmaceutical composition of claim 12, wherein the prolactin suppressor comprises a mutation in one of a prolactin receptor activation domain and a prolactin receptor heterodimerization domain.
